(19) United States
(12) Patent Application Publication
(10) Pub. No.: US 2017/0000977 A1
(43) Pub. Date: Jan. 5, 2017

(54) MEDICAL DEVICE HAVING OUTER POLYMERIC MEMBER INCLUDING ONE OR MORE CUTS

(71) Applicant: BOSTON SCIENTIFIC SCIMED, INC., Maple Grove, MN (US)

(72) Inventors: Gene T. Storbeck, Franklin, MA (US); Holly A. Kimball, Montrose, MN (US); David D. Groneberg, Plymouth, MN (US)

(21) Appl. No.: 15/196,946

(22) Filed: Jun. 29, 2016

Related U.S. Application Data
(60) Provisional application No. 62/186,920, filed on Jun. 30, 2015.

(51) Int. Cl. A61M 25/00; B29C 47/00; B29C 35/08; A61M 25/01

(52) U.S. Cl. CPC A61M 25/0054; A61M 25/0013; A61M 25/0045; A61M 25/01; A61M 25/0052; A61M 25/0108; B29C 47/0066; B29C 47/0023; B29C 35/08; B29C 47/0057; A61M 2025/0004; A61M 2025/0042; A61M 2025/0046; B29K 2105/02

(57) ABSTRACT

A medical device includes an outer polymeric tubular member and an inner tubular member. The outer polymeric tubular member includes one or more cuts formed therein to increase flexibility of the outer polymeric tubular member and includes an inner surface. The inner tubular member extends through the outer polymeric tubular member and has an outer surface in slidable contact with the inner surface of the outer polymeric tubular member. The outer polymeric tubular member is able to move relative to the inner tubular member as the medical device bends. In an example, the outer tubular member may be formed of a heat shrink material.

### CROSS REFERENCED TO RELATED APPLICATIONS

The present application claims the benefit of and priority to U.S. Provisional Patent Application Ser. No. 62/186,920, filed Jun. 30, 2015, the disclosure of which is incorporated herein by reference.

### TECHNICAL FIELD

The disclosure relates generally to medical devices. More specifically, the disclosure relates to medical devices such as catheters and the like, that include an elongate shaft and a reinforcing member disposed relative to the elongate shaft.

### BACKGROUND

A wide variety of medical devices have been developed for intracorporal use. Elongated medical devices are commonly used to facilitate navigation through and/or treatment within the anatomy of a patient. A variety of elongate medical devices such as catheters, endoscopes and the like have been developed over the past several decades. Because the anatomy of a patient may be tortuous, it is desirable to combine a number of performance features in such devices. For example, it is sometimes desirable that the device have a relatively high level of pushability and torqueability, particularly near its proximal end. It is also sometimes desirable that a device be relatively flexible, particularly near its distal end. A number of different elongated medical device structures and assemblies are known, each having certain advantages and disadvantages. However, there is an ongoing need to provide alternative elongated medical device structures and assemblies.

### SUMMARY

The disclosure provides design, material and manufacturing method alternatives for medical devices. An example medical device is disclosed. The medical device comprises:

an outer polymeric tubular member including one or more cuts formed therein to increase flexibility of the outer polymeric tubular member, the outer polymeric tubular member including an inner surface; and

an inner tubular member extending through the outer polymeric tubular member, the inner liner member having an outer surface in slidable contact with the inner surface of the outer polymeric tubular member;

wherein the outer polymeric tubular member is able to move relative to the inner tubular member when the medical device bends.

Alternatively or additionally to any of the embodiments above, the inner tubular member has an outer diameter, and the outer polymeric tubular member has an inner diameter that is less than about 0.001 inches larger than the outer diameter of the inner tubular member.

Alternatively or additionally to any of the embodiments above, the outer polymeric tubular member includes a cross-linked polymeric tube.

Alternatively or additionally to any of the embodiments above, the outer polymeric tubular member includes a heat shrink tubular member in a remembered configuration.

Alternatively or additionally to any of the embodiments above, the plurality of cuts in the outer polymeric tubular member are formed while the heat shrink tubular member is in an expanded-diameter expanded configuration.

Alternatively or additionally to any of the embodiments above, the inner tubular member is configured to be disposed within the outer polymeric tubular member while the heat shrink tubular member is in the expanded-diameter expanded configuration and is configured to be retained by the outer polymeric tubular member by converting the heat shrink tubular member to the remembered configuration.

Alternatively or additionally to any of the embodiments above, the polymeric tubular member polymeric tubular member is formed of a polymeric material having a flexural modulus in the range of about 100 MPa to about 4500 MPa.

Alternatively or additionally to any of the embodiments above, the plurality of cuts includes pairs of opposed slots.

Alternatively or additionally to any of the embodiments above, the inner tubular member includes a multilayer polymeric tube.

A catheter is disclosed. The catheter comprises:

an outer polymeric tubular member including a plurality of cuts formed therein to increase flexibility of the outer polymeric tubular member, the outer polymeric tubular member including an inner surface defining a lumen extending through the outer polymeric tubular member; and

an inner tubular member disposed within the lumen and extending through the outer polymeric tubular member, the inner tubular member having an outer surface in contact with the inner surface of the outer polymeric tubular member;

wherein the inner tubular member has an outer diameter, and the inner diameter of the outer polymeric tubular member is less than about 0.001 inches larger than the outer diameter of the inner tubular member.

Alternatively or additionally to any of the embodiments above, the outer polymeric tubular member includes a cross-linked polymeric tube having a remembered configuration and an expanded-diameter expanded configuration.

Alternatively or additionally to any of the embodiments above, the plurality of cuts in the outer polymeric tubular member are formed while the heat shrink tubular member is the expanded-diameter expanded configuration.

Alternatively or additionally to any of the embodiments above, the inner tubular member is configured to be disposed within the outer polymeric tubular member while the heat shrink tubular member is in the expanded-diameter expanded configuration and is configured to be retained by the outer polymeric tubular member once the heat shrink tubular member reverts to the remembered configuration.

Alternatively or additionally to any of the embodiments above, the polymeric tubular member polymeric tubular member is formed of a polymeric material having a flexural modulus in the range of about 100 MPa to about 4500 MPa.

Alternatively or additionally to any of the embodiments above, the inner tubular member includes a polymeric tube.

A method of forming a medical device is disclosed. The method comprises:

forming a plurality of cuts in at least a portion of a polymeric tubular member that is convertible between a remembered configuration and an expanded-diameter expanded configuration, the cuts being formed while the polymeric tubular member is in the expanded-diameter expanded configuration;

disposing an inner tubular member within the polymeric tubular member; and

converting the polymeric tubular member from the expanded configuration to the remembered configuration.

Alternatively or additionally to any of the embodiments above, forming a plurality of cuts includes laser cutting a plurality of cuts.

Alternatively or additionally to any of the embodiments above, forming a plurality of cuts includes forming a plurality of opposed pairs of slots.

Alternatively or additionally to any of the embodiments above, converting the polymeric tubular member from the expanded-diameter expanded configuration to the remembered configuration comprises heating the polymeric tubular member.

Alternatively or additionally to any of the embodiments above, the method further includes extruding a high modulus polymeric tubular member, cross-linking the high modulus polymeric tubular member to give it its remembered configuration, and then expanding the high modulus polymeric tubular member into its expanded-diameter expanded configuration.

The above summary of some embodiments is not intended to describe each disclosed embodiment or every implementation of the present invention. The Figures, and Detailed Description which follow more particularly exemplify these embodiments.

### BRIEF DESCRIPTION OF THE DRAWINGS

The invention may be more completely understood in consideration of the following detailed description of various embodiments of the invention in connection with the accompanying drawings, in which:

FIG. 1 is a side plan view of a medical device in accordance with one embodiment of the disclosure, shown as a guide or diagnostic catheter;

FIG. 2 is a partial longitudinal cross-sectional view of the medical device of FIG. 1;

FIG. 3 is a schematic perspective view of an inner tubular member, forming a portion of the medical device of FIG. 1; and

FIGS. 4 through 9 illustrate an exemplary manufacturing process for forming a portion of the medical device of FIG. 1.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (e.g., having the same function or result). In many instances, the terms “about” may include numbers that are rounded to the nearest significant figure.

Weight percent, percent by weight, wt %, wt-%, % by weight, and the like are synonyms that refer to the concentration of a substance as the weight of that substance divided by the weight of the composition and multiplied by 100.

The recitation of numerical ranges by endpoints includes all numbers within that range (e.g., 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following detailed description should be read with reference to the drawings in which similar elements in different drawings are numbered the same. The drawings, which are not necessarily to scale, depict illustrative embodiments and are not intended to limit the scope of the invention.

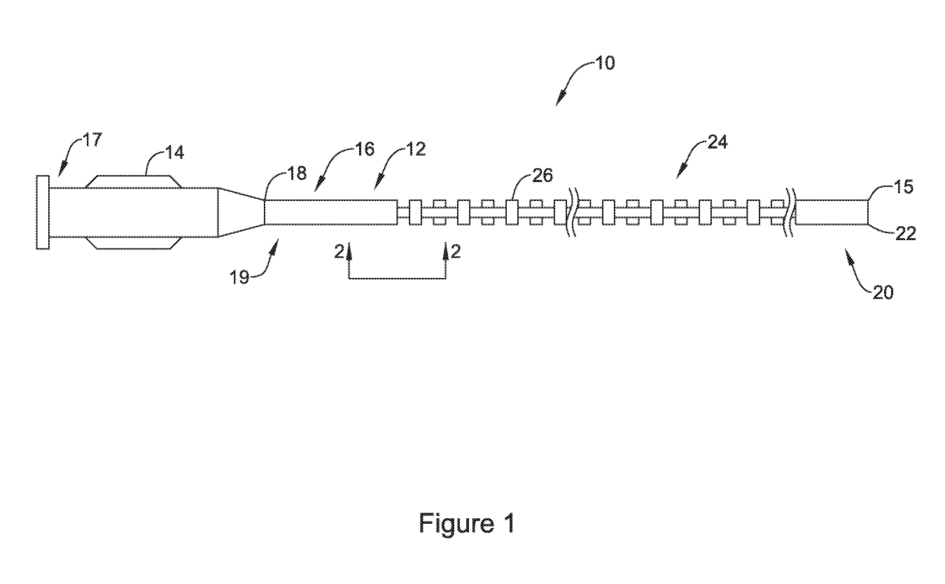

Refer now to FIG. 1 which illustrates a medical device 10 in accordance with one embodiment. In the embodiment shown, the medical device 10 is in the form of a guide or diagnostic catheter. Although set forth with specific reference to a guide or diagnostic catheter in the embodiments shown in the Figures and discussed below, the disclosure may relate to virtually any medical device including an elongate shaft or member having a reinforcing member disposed thereon. For example, the invention may be applied to medical devices such as a balloon catheter, an atherectomy catheter, a drug delivery catheter, a stent delivery catheter, an endoscope, an introducer sheath (if the sheath includes a reinforcing member), a fluid delivery device, other infusion or aspiration devices, device delivery (e.g., implantation) devices, and the like. Thus, while the Figures and descriptions below are directed toward a guide or diagnostic catheter, in other applications sizes in terms of diameter and length may vary widely, depending upon the desired properties of a particular device. For example, in some devices, lengths may range from about 1 centimeter (cm) to about 300 cm or more, while outside diameters may range from about 1 French (F) to about 20 F, or even more in some embodiments.

As shown in FIG. 1, the catheter 10 can include an elongate shaft 12 including a proximal portion 16 having a proximal end 18, and a distal portion 20 having a distal end 22. The shaft 12 is a generally tubular member defining a lumen 15 therein. A manifold 14 can be connected to the proximal end of the elongate shaft 12, and include a lumen and/or other structure to facilitate connection to other medical devices (e.g., syringe, Y-adapter, etc.) and to provide access to the lumen 15 within the shaft 12. The manifold 14 may include a hub portion 17 and a strain relief portion 19. In some embodiments, the shaft 12 may include additional devices or structures such as inflation or anchoring members, sensors, optical elements, ablation devices or the like, depending upon the desired function and characteristics of the catheter 10.

The guide or diagnostic catheter 10 may have a length and an outside diameter appropriate for its desired use, for example, to enable intravascular insertion and navigation. For example, the catheter 10 may have a length of about 20 cm to about 250 cm and an outside diameter of approximately 1 F to about 10 F, when catheter 10 is adapted as a guide catheter. In some embodiments, the catheter 10 can be a microcatheter that is adapted and/or configured for use within small anatomies of the patient. For example, some embodiments are particularly useful in treating targets located in tortuous and narrow vessels, for example in the neurovascular system, or in certain sites within the coronary vascular system, or in sites within the peripheral vascular system such as superficial femoral, popliteal, or renal arteries. The target site in some embodiments is a neurovascular site, such as site in the brain, which is accessible only via a tortuous vascular path, for example, a vascular path containing a plurality of bends or turns which may be greater than 90° turns, and/or involving vessels which are in the range of about 8 millimeters (mm) or less, and in some cases as small as 2 to 3 mm or less, in diameter. However, it is contemplated that the catheter may be used in other target sites within the anatomy of a patient. In some embodiments, the catheter can include an outside diameter in the range of about 1 F to about 4 F.

While in some embodiments, the catheter 10 can be described in terms of intravascular use, in other embodiments the guide or diagnostic catheter 10 may be suited for other uses in the digestive system, soft tissues, or any other use including insertion into an organism for medical uses. For example, in some embodiments, the catheter 10 may be significantly shorter and used as an introducer sheath, for example, while in other embodiments the catheter 10 may be adapted for other medical procedures. The guide or diagnostic catheter 10 may also include additional structure and materials that are substantially conventional.

Additionally, although depicted as including a generally round cross-sectional shape, it can be appreciated that the shaft 12 can include other cross-sectional shapes or combinations of shapes without departing from the spirit of the invention. For example, the cross-sectional shape of the generally tubular shaft 12 may be oval, rectangular, square, triangular, polygonal, and the like, or any other suitable shape, depending upon the desired characteristics.

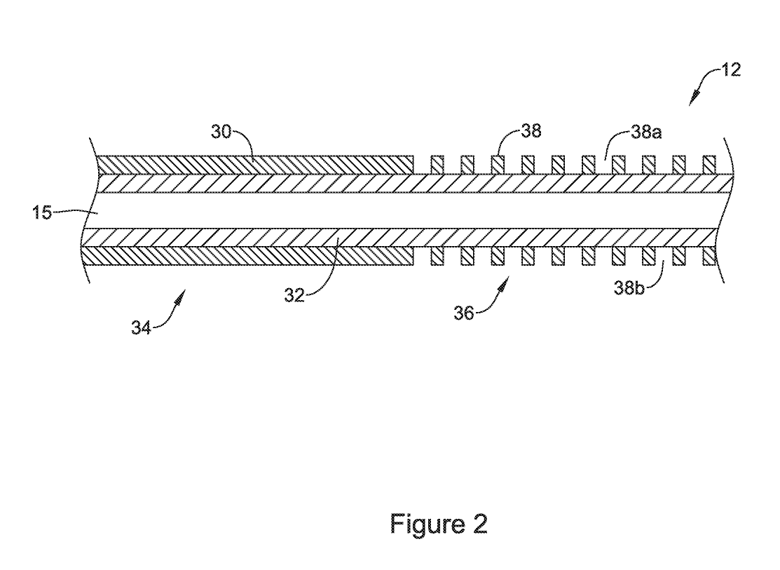

In some embodiments, the shaft 12 may include a section 24 that has one or more cuts 26. The cuts 26 may be formed along any desired portion of the shaft 12, and may increase the flexibility of the shaft 12 while retaining desirable strength characteristics. In some instances, at least some of the one or more cuts 26 extend all the way through the shaft wall while in other cases at least some of the cuts 26 may represent a thinning of the shaft wall. In some cases, as seen for example in FIG. 2, the shaft 12 may be a multiple layer construction. FIG. 2 is a cross-sectional view of a portion of the shaft 12 illustrating an outer tubular member 30 and an inner tubular member 32. In some instances, the inner tubular member 32 may be considered as forming or otherwise functioning as a liner within the outer tubular member 30. The outer tubular member 30 may include a portion 34 that does not include any slots or other cuts, and a portion 36 that includes cuts 38. While the cuts 38 are illustrated as being arranged as ordered pairs of slots 38a, 38b, it will be appreciated that the cuts 38 may take any desired shape. For example, the cuts 38 may include one or more cuts that wrap helically about the shaft 12.

As referenced in FIG. 1, the lumen 15, defined by the inner tubular member 32, may extend through the shaft 12. The lumen 15 can be adapted and/or configured to facilitate, for example, insertion of other medical devices (e.g., guide wires, balloon catheters, etc.) there through, and/or to facilitate injection of fluids (e.g., radiopaque dye, saline, drugs, inflation fluid, etc.) there through. The size of the lumen can vary, depending upon the desired characteristics and intended use. In some embodiments, the inner tubular member 32 can have an inner diameter, defining the lumen 15, that is in the range of about 0.01 to about 0.05 inch in size, and in some embodiments, in the range of about 0.015 to about 0.03 inch in size, and in some embodiments, in the range of about 0.016 to about 0.026 inch in size. Additionally, in some embodiments, the inner tubular member 32 can have an outer diameter that is in the range of about 0.011 to about 0.055 inch in size, and in some embodiments, in the range of about 0.015 to about 0.03 inch in size, and in some embodiments, in the range of about 0.019 to about 0.029 inch in size. It should be understood however, that these dimensions are provided by way of example only, and that in other embodiments, the size of the inner and outer diameter of the inner tubular member 32 can vary greatly from the dimensions given, depending upon the desired characteristics and function of the device. In some embodiments, the inner tubular member 32, or other portions of the shaft 12, can define one or more additional lumens depending upon the desired characteristics and function of the catheter 10, and such additional lumens can be shaped, size, adapted and/or configured the same as or different from lumen 15, depending upon the desired characteristic and functions.

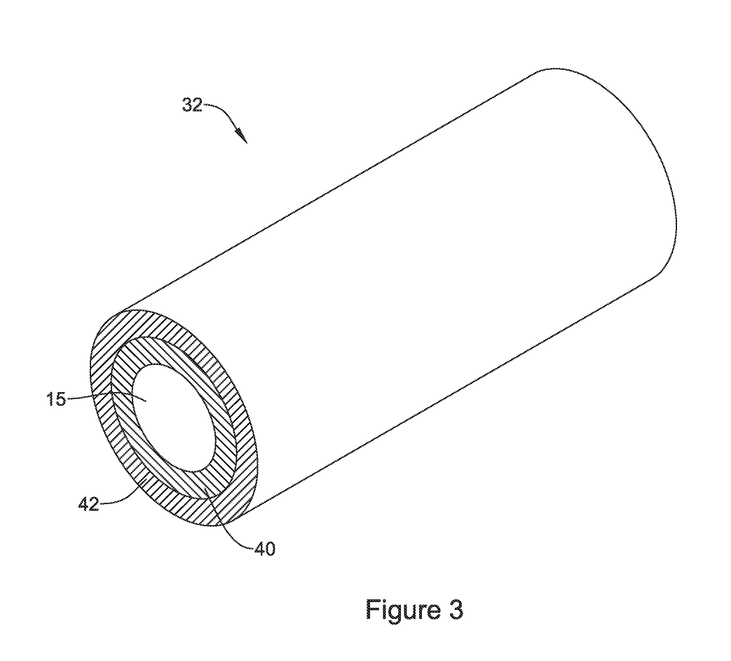

The inner tubular member 32, defining the lumen 15, may be one or more layers. As shown in FIG. 3, the inner tubular member 32 may be multi-layered. In the illustrative embodiment, the inner tubular member 32 may include an inner layer 40 and an outer layer 42. This is merely illustrative, as it should be understood that more or fewer layers can be used depending upon the desired characteristics of the device. Furthermore, while an inner layer 40 and an outer layer 42 are described with respect to the particular embodiment, these layers 40, 42 may be provided as a single layer. In some cases, for example, the layers 40, 42 may be co-extruded. In another example, the inner layer 40 and the outer layer 42 may be provided separately, but attached or combined together to physically form a single layer. One of the layers, for example, may include or otherwise provide a reinforcement such as a braid.

The inner layer 40 and the outer layer 42 may be made of any suitable material and by any suitable process, the materials and processes varying with the particular application. Examples of some suitable materials include, but are not limited to, polymers, metals, metal alloys, or composites or combinations thereof. Some examples of some suitable polymers can include, but are not limited to, polyoxymethylene (POM), polybutylene terephthalate (PBT), polyether block ester, polyether block amide (PEBA), fluorinated ethylene propylene (FEP), polyethylene (PE), polypropylene (PP), polyvinylchloride (PVC), polyurethane, polytetrafluoroethylene (PTFE), polyether-ether ketone (PEEK), polyimide, polyamide, polyphenylene sulfide (PPS), polyphenylene oxide (PPO), polysufone, nylon, perfluoro(propyl vinyl ether) (PFA), polyether-ester, polymer/metal composites, etc., or mixtures, blends or combinations thereof, and may also include or be made up of a lubricous polymer. One example of a suitable polyether block ester is available under the trade name ARNITEL, and one suitable example of a polyether block amide (PEBA) is available under the trade name PEBAX®, from ATOMCHEM POLYMERS, Birdsboro, Pa.

The inner layer 40 may include a lubricious polymer such as HDPE or PTFE, for example, or a copolymer of tetrafluoroethylene with perfluoroalkyl vinyl ether (PFA) (more specifically, perfluoropropyl vinyl ether or perfluoromethyl vinyl ether), or the like. The outer layer 42 may include a flexible polymer such as polyether block amide or polyether-ester elastomer. Additionally, in some embodiments, the polymer material of the inner layer 40 and/or the outer layer 42 can be blended with a liquid crystal polymer (LCP). For example, in some embodiments, the mixture can contain up to about 5% LCP. This has been found in some embodiments to enhance torqueability.

Additionally, as suggested above, in some embodiments, the inner tubular member 32 may include or be made of metal or metal alloys. Some examples of suitable metals and metal alloys can include stainless steel, such as 304V, 304L, and 316L stainless steel; nickel-titanium alloy such as a superelastic (i.e. pseudoelastic) or linear elastic nitinol; nickel-chromium alloy; nickel-chromium-iron alloy; cobalt alloy; tungsten or tungsten alloys; tantalum or tantalum alloys, gold or gold alloys, MP35-N (having a composition of about 35% Ni, 35% Co, 20% Cr, 9.75% Mo, a maximum 1% Fe, a maximum 1% Ti, a maximum 0.25% C, a maximum 0.15% Mn, and a maximum 0.15% Si); or the like; or other suitable metals, or combinations or alloys thereof. In some embodiments, it is desirable to use metals, or metal alloys that are suitable for metal joining techniques such as welding, soldering, brazing, crimping, friction fitting, adhesive bonding, etc.

The inner tubular member 32 can be formed by any suitable method or technique. For example in some embodiments, the inner layer 40 can be formed separately, and thereafter the outer layer 42 can be disposed thereon by suitable techniques, such as extrusion, co-extrusion, interrupted layer co-extrusion (ILC), coating, heat shrink techniques, casting, molding, or by fusing one or several segments of an outer layer material end-to-end about the inner layer 40, or the like. In some other embodiments, the layers 40, 42 may be formed together using suitable techniques, such as extrusion, co-extrusion, interrupted layer co-extrusion (ILC), heat shrink techniques, fusing, or the like. In yet other embodiments, the layers 40, 42 can be formed separately, such as by extrusion, co-extrusion, interrupted layer co-extrusion (ILC), casting, molding, heat shrink techniques, fusing, or the like, and thereafter coupled or connected together using suitable techniques, such as heat shrink techniques, friction fitting, mechanically fitting, chemically bonding, thermally bonding, welding (e.g., resistance, Rf, or laser welding), soldering, brazing, adhesive bonding, crimping, or the use of a connector member or material, or the like, or combinations thereof. In some instances, the inner tubular member 32 or some portion thereof may be formed via a 3D printing process.

The inner tubular member 32 may have a uniform stiffness, or may vary in stiffness along its length. For example, a gradual reduction in stiffness from the proximal end to the distal end thereof may be achieved, depending upon the desired characteristics. The gradual reduction in stiffness may be continuous or may be stepped, and may be achieved, for example, by varying the structure, such as the size or thickness of one or more of the layers 40, 42, or for example, by varying the materials used in one or more of the layers 40, 42. Such variability in characteristics and materials can be achieved, for example, by using techniques such as ILC, or by fusing together separate extruded tubular segments. Additionally, the inner layer 40 and/or the outer layer 42, may be impregnated with, or be made of or include a radiopaque material to facilitate radiographic visualization. Radiopaque materials are understood to be materials capable of producing a relatively bright image on a fluoroscopy screen or another imaging technique during a medical procedure. This relatively bright image aids the user of the catheter 10 in determining its location. Some examples of radiopaque materials can include, but are not limited to, gold, platinum, palladium, tantalum, tungsten alloy, polymer material loaded with radiopaque filler, and the like. Likewise, in some embodiments, the inner layer 40 and/or the outer layer 42 may be impregnated with, or be made of or include a material that may aid in MRI imaging. Some materials that exhibit these characteristics include, for example, tungsten, Elgiloy, MP35N, nitinol, and the like, and others. Those skilled in the art will recognize that these materials can vary widely without departing from the spirit of the invention.

Additionally, although depicted as including a generally round cross-sectional shape, it can be appreciated that the inner tubular member 32 can include other cross-sectional shapes or combinations of shapes without departing from the spirit of the invention. For example, the cross-sectional shape of the inner tubular member 32 may be oval, rectangular, square, triangular, polygonal, and the like, or any other suitable shape, depending upon the desired characteristics.

Returning to FIGS. 1 and 2, the outer tubular member 30 may be adapted and/or configured to have a desired level of stiffness, torqueability, flexibility, and/or other characteristics. Those of skill in the art and others will recognize that the dimensions, structure, and materials of the outer tubular member 30 are dictated primarily by the desired characteristics, and the function of the catheter 10, and that any of a broad range of the dimensions, structure, and materials can be used. The desired stiffness, torquability, lateral flexibility, bendability, or other such characteristics of the outer tubular member 30 can be imparted or enhanced by the structure of the outer tubular member 30. For example, the outer tubular member 30 may include a thin wall tubular structure, including one or moreof cuts 38, such as grooves, cuts, slits, slots, or the like, formed in a portion of, or along the entire length of, the outer tubular member 30. Such structure may be desirable because it may allow the outer tubular member 30, or portions thereof, to have a desired level of lateral flexibility as well as have the ability to transmit torque and pushing forces. The cuts 38 can be formed in essentially any known way. For example, the cuts 38 can be formed by methods such as micro-machining, saw-cutting, laser cutting, grinding, milling, casting, molding, chemically etching or treating, or other known methods, and the like. In some such embodiments, the structure of the outer tubular member 30 is formed by cutting and/or removing portions of the tube to form the cuts 38.

The outer tubular member 30 may be formed of any desired materials. In some cases, as will be discussed, the outer tubular member 30 is formed of a heat shrink material. As is known, a heat shrink tube can be formed by extruding a starting tube from a raw material including the desired polymer, and possibly including additives such as colorants, stabilizers and the like. The starting tube is then subjected to a cross-linking process, such as with radiation although other cross-linking processes are contemplated. In some cases, this cross-linking creates a memory in the tube. At this stage, the extruded diameter (or other size measurement) may be considered as the remembered diameter (or other size measurement) of the tube. The tube is heated to just above the crystalline melting point of the polymer and is expanded in diameter. In some instances, this expanded diameter may be considered as the expanded-diameter expanded configuration of the heat shrink tube. In some cases, the heat shrink tube is expanded by exposing the heat shrink tube to vacuum, followed by rapid cooling. Subsequently, when heated again to above the crystalline melting point of the polymer, the heat shrink tube reverts back to its remembered configuration including the remembered diameter, or originally extruded size.

A variety of heat shrink polymers may be utilized in forming the outer tubular member 30. In some cases, the outer tubular member 30 may be formed of a polymer or blend of polymers that has a relative high flexural modulus. In some cases, the outer tubular member 30 is formed of one or more polymers having a modulus that is in the range of about 100 MPa to about 4500 MPa (4.5 GPa). Illustrative but non-limiting examples of suitable polymers include PEEK (polyetheretherketone), PEK (polyetherketone), PEKK (polyetherketoneketone), PAEK (polyaryletherketone), PET (polyethylphthlalate), PE (polyethylene), PEBA (polyetherblockamide), various nylons. Illustrative but non-limiting examples of suitable fluoropolymers include PTFE (polytetrafluoroether), FEP (fluorinatedethylenepropylene) and PFA (perfluoralkoxy). In some instances, filled polymers may also be used. In some embodiments, PE may have a flexural modulus of about 400 MPa to about 1500 MPA. In some instances, nylons may have a flexural modulus of about 1 GPa to about 2 GPa. PEBA may, for example, have a flexural modulus of about 100 MPa to about 500 MPa. In some instances, PEKK may have a flexural modulus of about 2 GPa to at least 4 GPa.

In some embodiments, the cuts 38 may be formed in the outer tubular member 30 while the outer tubular member 30 is in its expanded-diameter expanded configuration. Various embodiments of arrangements and configurations of slots are also contemplated that may be used in addition to what is described above or may be used in alternate embodiments. For simplicity purposes, the following disclosure makes reference to the catheter 10, the cuts (slots) 38, and the outer tubular member 30 referenced in relation to FIG. 1 and FIG. 2. However, it can be appreciated that these variations may also be utilized for other slots and/or tubular members. In some embodiments, at least some, if not all, of the slots 38 are disposed at the same or a similar angle with respect to a longitudinal axis of the outer tubular member 30. As shown, the slots 38 can be disposed at an angle that is perpendicular, or substantially perpendicular, and/or can be characterized as being disposed in a plane that is normal to the longitudinal axis of the outer tubular member 30. However, in other embodiments, the slots 38 can be disposed at an angle that is not perpendicular, and/or can be characterized as being disposed in a plane that is not normal to the longitudinal axis of the outer tubular member 30. Additionally, a group of one or more slots 38 may be disposed at different angles relative to another group of one or more slots 38. The distribution and/or configuration of slots 38 can also include, to the extent applicable, any of those disclosed in U.S. Pat. Publication No. US 2004/0181174, the entire disclosure of which is herein incorporated by reference.

The slots 38 may be provided to enhance the flexibility of the outer tubular member 30 while still allowing for suitable torque transmission characteristics. The slots 38 may be formed such that one or more rings and/or tube segments interconnected by one or more segments and/or beams that are formed in the outer tubular member 30, and such tube segments and beams may include portions of the outer tubular member 30 that remain after slots 38 are formed. Such an interconnected structure may act to maintain a relatively high degree of torsional stiffness, while maintaining a desired level of lateral flexibility. In some embodiments, some adjacent slots 38 can be formed such that they include portions that overlap with each other about the circumference of the outer tubular member 30. In other embodiments, some adjacent slots 38 can be disposed such that they do not necessarily overlap with each other, but are disposed in a pattern that provides the desired degree of lateral flexibility.

Additionally, the slots 38 can be arranged along the length of, or about the circumference of, the outer tubular member 30 to achieve desired properties. For example, adjacent slots 38, or groups of slots 38, can be arranged in a symmetrical pattern, such as being disposed essentially equally on opposite sides about the circumference of the outer tubular member 30, or can be rotated by an angle relative to each other about the axis of the outer tubular member 30. Additionally, adjacent slots 38, or groups of slots 38, may be equally spaced along the length of the outer tubular member 30, or can be arranged in an increasing or decreasing density pattern, or can be arranged in a non-symmetric or irregular pattern. Other characteristics, such as slot size, slot shape, and/or slot angle with respect to the longitudinal axis of the outer tubular member 30, can also be varied along the length of the outer tubular member 30 in order to vary the flexibility or other properties. In other embodiments, moreover, it is contemplated that the portions of the outer tubular member 30, such as a proximal section, or a distal section, or the entire outer tubular member 30, may not include any such slots 38.

As suggested herein, the slots 38 may be formed in groups of two, three, four, five, or more slots 38, which may be located at substantially the same location along the axis of the outer tubular member 30. Alternatively, a single slot 38 may be disposed at some or all of these locations. Within the groups of slots 38, there may be included slots 38 that are equal in size (e.g., span the same circumferential distance around the outer tubular member 30). In some of these as well as other embodiments, at least some slots 38 in a group are unequal in size (e.g., span a different circumferential distance around the outer tubular member 30). Longitudinally adjacent groups of slots 38 may have the same or different configurations. For example, some embodiments of outer tubular member 30 include slots 38 that are equal in size in a first group and then unequally sized in an adjacent group. It can be appreciated that in groups that have two slots 38 that are equal in size and are symmetrically disposed around the tube circumference, the centroid of the pair of beams (e.g., the portion of the outer tubular member 30 remaining after slots 38 are formed therein) is coincident with the central axis of the outer tubular member 30. Conversely, in groups that have two slots 38 that are unequal in size and whose centroids are directly opposed on the tube circumference, the centroid of the pair of beams can be offset from the central axis of the outer tubular member 30. Some embodiments of the outer tubular member 30 include only slot groups with centroids that are coincident with the central axis of the outer tubular member 30, only slot groups with centroids that are offset from the central axis of outer tubular member 30, or slot groups with centroids that are coincident with the central axis of outer tubular member 30 in a first group and offset from the central axis of outer tubular member 30 in another group. The amount of offset may vary depending on the depth (or length) of slots 38 and can include other suitable distances.

The slots 38 can be formed by methods such as micro-machining, saw-cutting (e.g., using a diamond grit embedded semiconductor dicing blade), electron discharge machining, grinding, milling, casting, molding, chemically etching or treating, or other known methods, and the like. In some such embodiments, the structure of the outer tubular member 30 is formed by cutting and/or removing portions of the tube to form the slots 38. Some examples of appropriate micromachining methods and other cutting methods, and structures for tubular members including slots and medical devices including tubular members are disclosed in U.S. Pat. Publication Nos. 2003/0069522 and 2004/0181174-A2; and U.S. Pat. Nos. 6,766,720; and 6,579,246, the entire disclosures of which are herein incorporated by reference. Some examples of etching processes are described in U.S. Pat. No. 5,106,455, the entire disclosure of which is herein incorporated by reference.

In at least some embodiments, the slots 38 may be formed in the tubular member using a laser cutting process. The laser cutting process may include a suitable laser and/or laser cutting apparatus. For example, the laser cutting process may utilize a fiber laser. Utilizing processes like laser cutting may be desirable for a number of reasons. For example, laser cutting processes may allow the outer tubular member 30 to be cut into a number of different cutting patterns in a precisely controlled manner. This may include variations in the slot width, ring width, beam height and/or width, etc. Furthermore, changes to the cutting pattern can be made without the need to replace the cutting instrument (e.g., blade). This may also allow smaller tubes (e.g., having a smaller outer diameter) to be used to form the outer tubular member 30 without being limited by a minimum cutting blade size. Consequently, the outer tubular member 30 may be fabricated for use in neurological devices or other devices where a relatively small size may be desired.

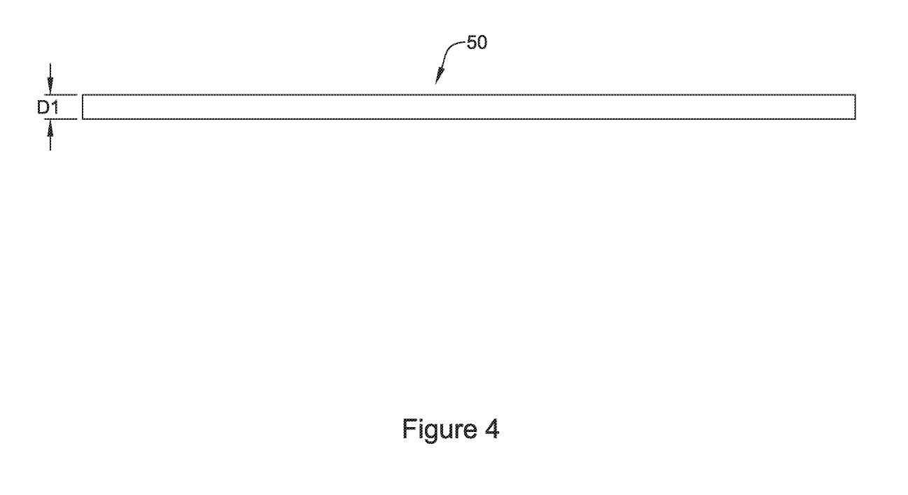

FIGS. 4 through 9 illustrate an exemplary process by which the outer tubular member 30 and the inner tubular member 32 may be assembled together to provide a portion of the shaft 12 of the catheter 10. It will be appreciated that the outer tubular member 30 and the inner tubular member 32, in combination, only form a portion of the catheter 12. Additional components, such as the manifold 14 or additional structures (not illustrated) added to the distal tip may be added in any desired or conventional manner.

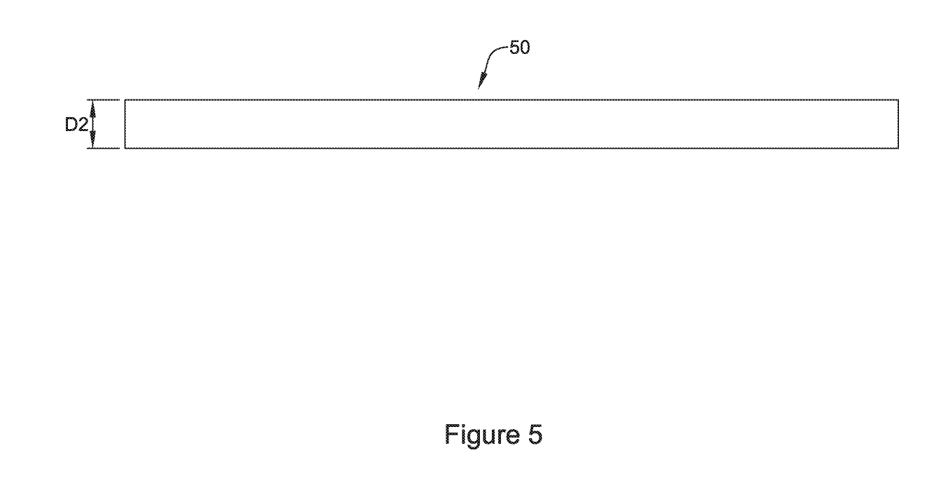

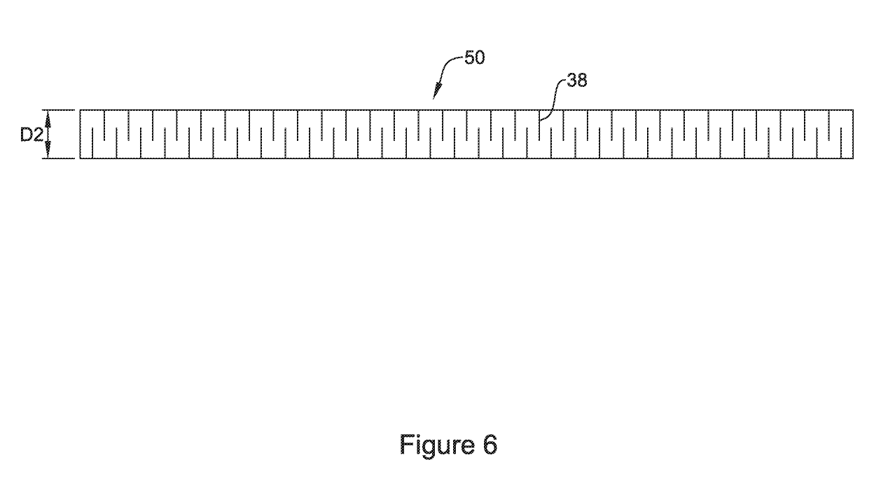

FIG. 4 illustrates a polymer tube 50, sized as extruded. The polymer tube 50 may be formed of any suitable heat shrink material, such as those discussed above. As illustrated, the polymer tube 50 may have already been subjected to a cross-linking and thermal processing to instill its remembered configuration. It can be seen that the polymer tube 50 has a diameter D1 which may be in the range of about 0.01 inches (0.25 mm) to about 0.5 inches (12.7 mm), depending on the desired functionality of the catheter 10. In an example, the diameter D1 is the remembered diameter. In FIG. 5, the polymer tube 50 can be seen as having been expanded to a larger diameter D2 (relative to the remembered diameter D1) which may be in the range of about 0.02 inches (0.51 mm) to about 1 inch (25.4 mm). In an example, the diameter D2 is the expanded diameter of the polymer tube 50 in the expanded-diameter expanded configuration. This may occur, for example, by having subjected the polymer tube 50 to a vacuum source, as discussed above. While still expanded, the slots 38 are cut into the polymer tube 50 as seen in FIG. 6. As noted above, the slots 38 may take any particular or desired pattern and dimensions.

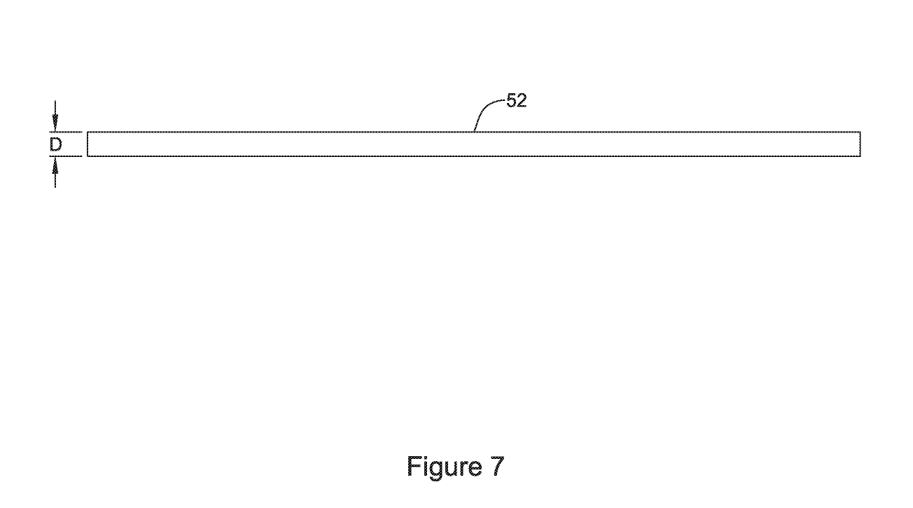

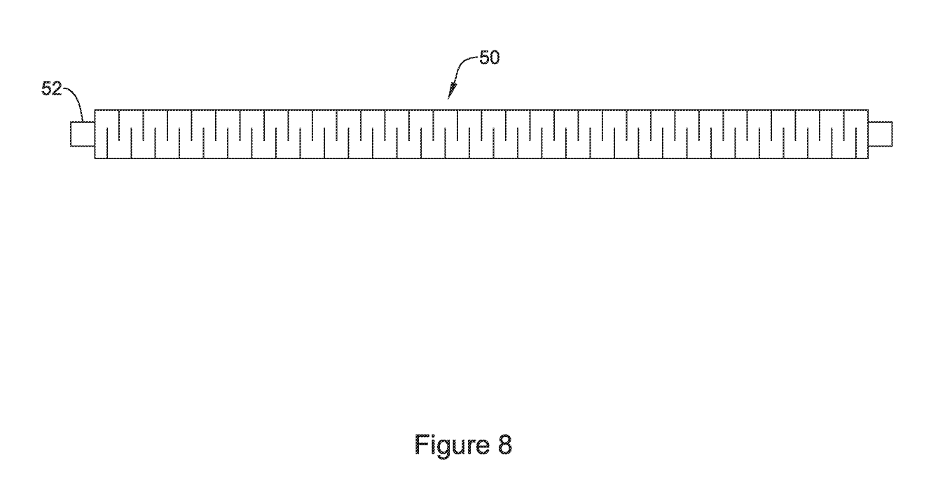

FIG. 7 illustrates a polymer tube 52 that forms the inner tubular member 32 (FIG. 1). The polymer tube 52 may be formed of any desired material, such as those discussed above, and may have a diameter D. The polymer tube 52 may be disposed within the polymer tube 50, once cut and still in its expanded configuration, as seen in FIG. 8. In the expanded configuration, the polymer tube 50 may have an inner diameter that is at least about 0.001 inches (0.025 mm) larger than an outer diameter of the polymer tube 52 such that the polymer tube 52 may easily be disposed within the polymer tube 50.

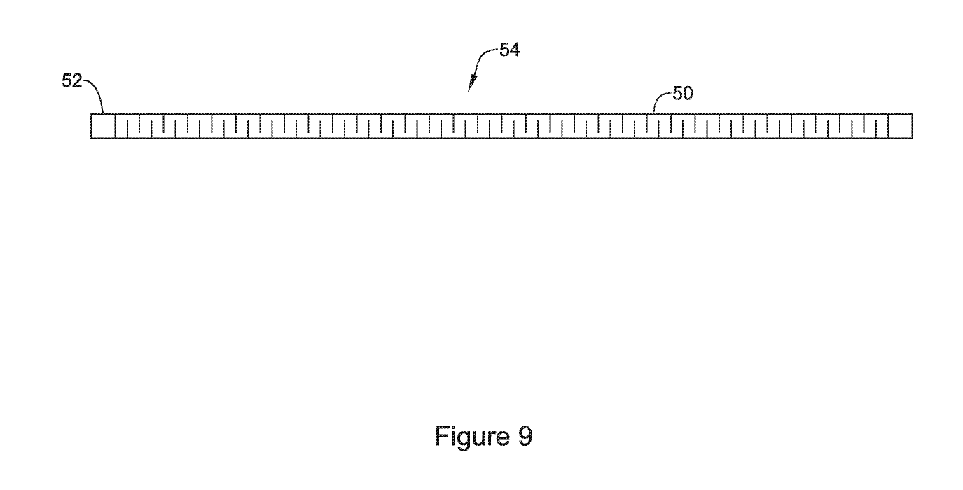

Applying heat may cause the polymer tube 50 to revert to its remembered configuration, as shown in FIG. 9, forming a composite structure 54 which can then be used to form the rest of the catheter 10. It will be appreciated that by controlling the outer diameter of the polymer tube 52, and the inner diameter of the originally extruded (or remembered) configuration of the polymer tube 50, it is possible to provide a composite structure in which the outer tubular member 30 is in intimate contact with the inner tubular member 32. The outer tubular member 30 may be permitted to slide or otherwise move relative to the inner tubular member 32 when the resultant catheter 10 is curved or flexed. In some instances, the inner tubular member 32 may be considered as being retained by the outer tubular member 30 once the outer tubular member 32 is in its remembered configuration.

In comparison to previous structures in which a polymeric liner is inserted into a metallic reinforcing member, there is virtually no wasted space between the outer tubular member 30 and the inner tubular member 32. For example, the outer tubular member 30 may have an inner diameter, once the outer tubular member 30 has been converted back to its original diameter, that is less than about 0.001 inches (0.025 mm) larger than an outer diameter of the inner tubular member 32. Accordingly, for a given outer diameter of the catheter 10, the lumen 15 may have a relatively larger inner diameter. Put another way, for a given inner diameter of the lumen 14, the catheter 10 may have a relatively smaller outer diameter. Accordingly, use of a heat shrink tube for creating the outer tubular member 30 provides manufacturing advantages.

A lubricious, a hydrophilic, a protective, or other type of coating may be applied over portions or all of the shaft 12. Hydrophobic coatings such as fluoropolymers provide a dry lubricity which improves catheter handling and device exchanges. Lubricious coatings can aid in insertion and steerability. Suitable lubricious polymers are well known in the art and may include silicone and the like, hydrophilic polymers such as polyarylene oxides, polyvinylpyrolidones, polyvinylalcohols, hydroxy alkyl cellulosics, algins, saccharides, caprolactones, and the like, and mixtures and combinations thereof. Hydrophilic polymers may be blended among themselves or with formulated amounts of water insoluble compounds (including some polymers) to yield coatings with suitable lubricity, bonding, and solubility. Some other examples of such coatings and materials and methods used to create such coatings can be found in U.S. Pat. Nos. 6,139,510 and 5,772,609, which are incorporated herein by reference.

It should also be understood that in some embodiments, a degree of MRI compatibility can be imparted into the catheter 10. For example, to enhance compatibility with Magnetic Resonance Imaging (MRI) machines, it may be desirable to construct various portions of the catheter 10, in a manner, or use materials that would impart, a degree of MRI compatibility. For example, the lengths of relatively conductive structures within the catheter 10 may be limited to lengths that would not generate undue heat due to resonance waves created in such structures when under the influence of an MRI field generated by an MRI machine. Alternatively, or additionally, portions, or all of the catheter may be made of a material that does not substantially distort the image and create substantial artifacts (artifacts are gaps in the image). Certain ferromagnetic materials, for example, may not be suitable because they may create artifacts in an MRI image. Additionally, all or portions of the catheter may also be made from a material that the MRI machine can image, as described above. Some materials that exhibit these characteristics include, for example, tungsten, Elgiloy, MP35N, nitinol, and the like, and others.

The present invention should not be considered limited to the particular examples described above, but rather should be understood to cover all aspects of the invention as fairly set out in the attached claims. Various modifications, equivalent processes, as well as numerous structures to which the present invention may be applicable will be readily apparent to those of skill in the art to which the present invention is directed upon review of the instant specification. It should be understood that this disclosure is, in many respects, only illustrative. Changes may be made in details, particularly in matters of shape, size, and arrangement of steps without exceeding the scope of the invention. The scope of the invention is, of course, defined in the language in which the appended claims are expressed.

We claim:

1. A medical device comprising:
an outer polymeric tubular member including one or more cuts formed therein to increase flexibility of the outer polymeric tubular member, the outer polymeric tubular member including an inner surface; and
an inner tubular member extending through the outer polymeric tubular member, the inner tubular member having an outer surface in slidable contact with the inner surface of the outer polymeric tubular member;
wherein the outer polymeric tubular member is able to move relative to the inner tubular member when the medical device bends.

2. The medical device of claim 1, wherein the inner tubular member has an outer diameter, and the outer polymeric tubular member has an inner diameter that is less than about 0.001 inches larger than the outer diameter of the inner tubular member.

3. The medical device of claim 1, wherein the outer polymeric tubular member includes a cross-linked polymeric tube.

4. The medical device of claim 1, wherein the outer polymeric tubular member includes a heat shrink tubular member in a remembered configuration.

5. The medical device of claim 4, wherein the one or more cuts in the outer polymeric tubular member are formed while the heat shrink tubular member is in an expanded configuration.

6. The medical device of claim 5, wherein the inner tubular member is configured to be disposed within the outer polymeric tubular member while the heat shrink tubular member is in the expanded configuration and is configured to be retained with in the outer polymeric tubular member by converting the heat shrink tubular member to the remembered configuration.

7. The medical device of claim 1, wherein the polymeric tubular member is formed of a polymeric material having a flexural modulus in the range of about 100 MPa to about 4500 MPa.

8. The medical device of claim 1, wherein the one or more cuts includes pairs of opposed slots.

9. The medical device of claim 1, wherein the inner tubular member includes a multilayer polymeric tube.

10. A catheter comprising:
an outer polymeric tubular member including a plurality of cuts formed therein to increase flexibility of the outer polymeric tubular member, the outer polymeric tubular member including an inner surface defining a lumen extending through the outer polymeric tubular member; and
an inner tubular member disposed within the lumen and extending through the outer polymeric tubular member, the inner tubular member having an outer surface in contact with the inner surface of the outer polymeric tubular member;
wherein the inner tubular member has an outer diameter, and the inner diameter of the outer polymeric tubular member is less than about 0.001 inches larger than the outer diameter of the inner tubular member.

11. The catheter of claim 10, wherein the outer polymeric tubular member includes a cross-linked polymeric tube having a remembered configuration and an expanded-diameter expanded configuration.

12. The catheter of claim 11, wherein the plurality of cuts in the outer polymeric tubular member is formed while the heat shrink tubular member is the expanded-diameter expanded configuration.

13. The catheter of claim 12, wherein the inner tubular member is configured to be disposed within the outer polymeric tubular member while the heat shrink tubular member is in the expanded-diameter expanded configuration and is configured to be retained within the outer polymeric tubular member once the heat shrink tubular member reverts to the remembered configuration.

14. The catheter of claim 11, wherein the polymeric tubular member is formed of a polymeric material having a flexural modulus in the range of about 100 MPa to about 4500 MPa.

15. The catheter of claim 11, wherein the inner tubular member includes a polymeric tube.

16. A method of forming a medical device, the method comprising:
forming a plurality of cuts in at least a portion of a polymeric tubular member that is convertible between a remembered configuration and an expanded-diameter expanded configuration, the cuts being formed while the polymeric tubular member is in the expanded-diameter expanded configuration;
disposing an inner tubular member within the polymeric tubular member; and
converting the polymeric tubular member from the expanded-diameter expanded configuration to the remembered configuration.

17. The method of claim 16, wherein forming a plurality of cuts includes laser cutting a plurality of cuts.

18. The method of claim 16, wherein forming a plurality of cuts includes forming a plurality of opposed pairs of slots.

19. The method of claim 16, wherein converting the polymeric tubular member from the expanded-diameter expanded configuration to the remembered configuration includes heating the polymeric tubular member.

20. The method of claim 16, further comprising extruding a high modulus polymeric tubular member, cross-linking the high modulus polymeric tubular member to give it its remembered configuration, and then expanding the high modulus polymeric tubular member into its expanded-diameter expanded configuration.
